(19) United States
(12) Patent Application Publication
(10) Pub. No.: US 2015/0245859 A1
(43) Pub. Date: Sep. 3, 2015

(54) Cervical Plate With Retaining Clip

(71) Applicant: DEROYAL INDUSTRIES, INC., Powell, TN (US)

(72) Inventors: Troy Allen McMillen, Knoxville, TN (US); Nicholas Poker, Knoxville, TN (US); Travis Lane Turley, Knoxville, TN (US); Nephi Zufelt, Knoxville, TN (US)

(21) Appl. No.: 14/432,779

(22) PCT Filed: Oct. 15, 2013
(86) PCT No.: PCT/US13/65072

§ 371 Date: Apr. 1, 2015

Related U.S. Application Data
(60) Provisional application No. 61/715,960, filed on Oct. 19, 2012.

(51) Int. Cl. A61B 17/80

(52) U.S. Cl. CPC A61B 17/8033; A61B 17/808

(57) ABSTRACT

A cervical plate apparatus includes a cervical plate body having a plurality of screw receiving passages defined therethrough. Received about each of the passages is a zero-step locking member which is operatively associated with the passage and configured to automatically lock the head of the screw in place within the passage when the screw is seated within the passage to attach the cervical plate to the cervical vertebrae. The locking member is selectively movable to an unlocked position. A retention structure is configured to retain the locking member in either the unlocked or locked position without further engagement of the locking member by a surgeon, so that when the locking member has been moved to the unlocked position, it is held in that position so that the surgeon can then remove the screw or screws without having to simultaneously hold the locking member in a retracted position.

### TECHNICAL FIELD

The present invention relates generally to apparatus for use in the connection of orthopedic implants to bones, and more specifically, but not by way of limitation, to apparatus for use in the fusion of vertebral bodies of the cervical spine, and to methods of using such apparatus.

### BACKGROUND ART

Fusion is a surgical treatment for clinical conditions such as Degenerative Disk Disease (DDD), deformity, trauma or other conditions. The pertinent anatomical cervical vertebral element of interest to this invention is the body portion of the vertebrae comprised of an outer cortical bone “can like” shell and an inner cancellous bone core. The top and bottom “lids” of this cortical shell are known as the endplates. During this surgical treatment the cartilaginous connection structure, known as the disk, between adjacent vertebrae is removed and a graft of bone is applied to maintain or restore the proper spacing between the vertebrae. When fusing cervical vertebrae it is necessary to immobilize the endplates where fusion is desired with respect to one another to foster a proper bone growth environment. Excessive motion between the endplates and the graft will inhibit fusion and the long term effectiveness of the treatment would be compromised.

The use of cervical plates and screws to provide this relative immobilization between the vertebral bodies is well established within the clinical history of this surgical treatment. One of the problems with the historical designs was retention of the screws within the plate to maintain immobilization. Over the course of this treatments history, several screw retention methods have been employed such as additional retainer plates applied over the screw heads to prevent back out, expanding screw heads, or displacement capture mechanisms where a tab is deformed or indexed into a locking position after the screw has been placed. The commonality between these locking mechanisms is that they all required additional steps by the surgeon to establish the locked or retaining condition of the plate. These additional steps are difficult and time consuming for a surgeon to perform due to limited visibility and access to the plate implantation site on the anterior cervical vertebral column. The access aperture is only approximately 1.5″ diameter by 4″ deep.

In the most recent history of this surgical treatment, the designs have evolved to minimize or eliminate the steps required to actuate the locking mechanisms for the screws. The problem with these so called “zero-step” designs is that they do not accommodate for the scenarios where the surgeon must remove and/or replace the screws due to screw mal-alignment, screw to bone strip out, or other routine surgical scenarios. In these scenarios, the surgeons must pry open the locking mechanisms and remove the screws simultaneously within the limited aperture and this further limits the visibility available to perform the task.

### DISCLOSURE OF THE INVENTION

The present invention provides a cervical plate with a locking mechanism that eliminates the need for additional locking steps while allowing for a selectable locking actuation of the plate to facilitate desired screw removal. The locking mechanism does not require simultaneous locking mechanism actuation and screw removal.

In one embodiment an orthopedic implant apparatus includes an implant body having at least one screw receiving hole defined through the implant body. A resilient locking clip is connected to the implant body and selectively movable relative to the implant body between an actuated position wherein the clip automatically prevents screw back-out from the hole after screw insertion into the hole, and an unactuated position wherein the clip is displaced relative to the hole so that the screw may be removed from the hole. A retention assembly includes an implant retention part connected to the implant body and a clip retention part connected to the clip. The implant retention part and the clip retention part are configured to hold the clip in either selected one of the actuated and unactuated positions relative to the implant body.

In another embodiment an orthopedic implant apparatus includes a plate body and a resilient clip. The plate body includes an outward body surface, an inward body surface, and first and second screw receiving holes extending through the plate body from the outward body surface to the inward body surface for receiving first and second screws, respectively. A central area of the plate body is located between the first and second holes, and has a first locking member defined thereon. A first undercut groove at least partially surrounds the first hole adjacent the outward body surface. A second undercut groove at least partially surrounds the second hole adjacent the outward body surface. The resilient clip is received in the first and second undercut grooves and spans the central area. The clip includes a first open loop portion received in the first undercut groove and including a first free end. The clip further includes a second open loop portion received in the second undercut groove and including a second free end. The clip further includes an intermediate clip portion joining the first and second open loop portions and spanning the central area. The intermediate clip portion includes a second locking member defined thereon and operatively engaged with the first locking member so that the clip is movable relative to the plate body between a locked position wherein the first and second open loop portions extend laterally into the first and second holes to prevent the screws from backing out of the holes, and an unlocked position wherein the first and second open loop portions are displaced laterally outward from the first and second holes so that the clip does not interfere with removal of the screws.

In another embodiment an orthopedic implant apparatus includes an implant body having at least one screw receiving passage defined therethrough. The passage has a reduced diameter socket end. A screw having an elongated portion and an enlarged head is configured to seat on the socket end of the passage. A zero-step locking member is connected to the implant body and operatively associated with the passage and configured to automatically lock the head of the screw in place in the passage when the head of the screw is seated on the socket end. The locking member is selectively movable to an unlocked position. A retention structure is configured to retain the locking member in the unlocked position without further engagement of the locking member by a surgeon so that the screw can be removed after the locking member is moved to the unlocked position.

In another embodiment a method is provided for attaching an orthopedic implant body to a bone. The method comprises:

(a) inserting a screw through an opening of the implant body and into the bone to seat the screw on the implant body and thus attach the implant body to the bone;

(b) automatically locking the screw in the implant body during step (a) by movement of a resilient retaining clip as the screw is inserted past the retaining clip, and thereby preventing back-out of the screw, without any additional locking action being necessary by a surgeon performing the method;

(c) moving the retaining clip to an unlocked position wherein the retaining clip is held in the unlocked position by a detent structure of the implant body; and

(d) after step (c), removing the screw without the surgeon having to simultaneously retract the retaining clip.

In each of the embodiments above the orthopedic implant apparatus may be a cervical plate for joining two cervical vertebrae as part of a cervical fusion operation. The cervical plate may include first and second pairs of such holes through the plate, and the clip or locking member includes two such clips or locking members, each of which is operable to lock two screws in two adjacent holes.

In each of the embodiments described above, the clip or locking member may be a w-shaped resilient member.

In each of the embodiments above the clip or locking member may be resiliently biased laterally relative to the hole through the implant body when the clip or locking member is in its actuated position, and the implant body may include a cam surface for guiding laterally outward movement of the clip when the clip is moved from its actuated to its unactuated position.

The clip may either be resiliently biased laterally inward or laterally outward relative to its associated hole, and the associated cam surface is formed in a complementary manner to control the laterally outward movement of the clip structure as the clip is moved from its actuated to its unactuated position.

In each of the embodiments described above the portion of the clip associated with each hole preferably includes a plurality of interference tabs extending laterally inward toward the associated hole, and the plurality preferably includes at least three such tabs.

In each of the described embodiments the clip is preferably received in an undercut groove surrounding its associated hole or holes.

The clips are preferably integrally formed from a nitinol material.

Numerous objects, features and advantages of the present invention will be readily apparent to those skilled in the art upon a reading of the following disclosure when taken in conjunction with the accompanying drawings.

### BRIEF DESCRIPTION OF THE DRAWINGS

FIG. 1 is a schematic elevation view showing a cervical plate in position relative to two cervical vertebrae which are shown in phantom lines.

FIG. 2 is a front elevation view of the cervical plate.

FIG. 3 is a right side elevation view of the cervical plate of FIG. 2.

FIG. 4 is a bottom view of the cervical plate of FIG. 2.

FIG. 5 is a schematic cross-section view taken along line 5-5 of FIG. 1 and showing the relationship of the cervical plate to the cervical vertebrae with one screw in place anchoring the cervical plate to the cervical vertebrae.

FIG. 6 is a perspective upper end of the screw showing the drive socket formed in the upper end of the enlarged head of the screw.

FIG. 7 is an elevation view of one of the retaining clips in a relaxed position.

FIG. 8 is a schematic front elevation view of the cervical plate cut away to show the profile of the undercut grooves, with a spring clip in relaxed position superimposed over its approximate location within the cervical plate.

FIG. 9 is a view similar to FIG. 8, but showing the upper half cut away with the spring clip in its actuated position with the free arms of the clip trapped within the undercut grooves and held in a resiliently outwardly biased position. The cut away of FIG. 9 is made generally along the line 9-9 shown in FIG. 5, with the cut generally parallel to the curved planar front surface of the plate. FIG. 9 also shows the lower half not cut away, and shows a second clip in place around the lower pair of holes.

FIG. 10 is a view similar to FIG. 9, showing the upper spring clip moved axially downward to its unactuated position, and the lower spring clip moved axially upward to its unactuated position, with the free arms of the spring clips having been guided laterally outward away from the holes by the cam surfaces of the undercut grooves.

FIG. 11 is a view similar to FIG. 9 illustrating one of the screws in place with its enlarged head located below the upper clip which is in the actuated position to retain the screw in place within the cervical plate.

FIG. 12 illustrates an actuating tool utilized to move the spring clip between its actuated and unactuated positions relative to the cervical plate.

FIG. 13 is a view similar to FIG. 9, illustrating an alternative embodiment.

### BEST MODE FOR CARRYING OUT THE INVENTION

Referring now to the drawings, and particularly to FIGS. 8-11, a cervical plate assembly, which may be more generally referred to as an orthopedic implant apparatus, is shown and generally designated by the numeral 10. The apparatus 10 includes a cervical plate 12 which may be more generally referred to as an implant body or plate body 12. The apparatus further includes first and second resilient locking clips 14 and 15 which may also be referred to as locking members 14 and 15. The apparatus 10 further includes a retention assembly 16 including a first retention structure 18 defined on the cervical plate 12 and a second retention structure 20 defined on the clip 14. The retention assembly 16 may also alternatively be referred to as a detent structure.

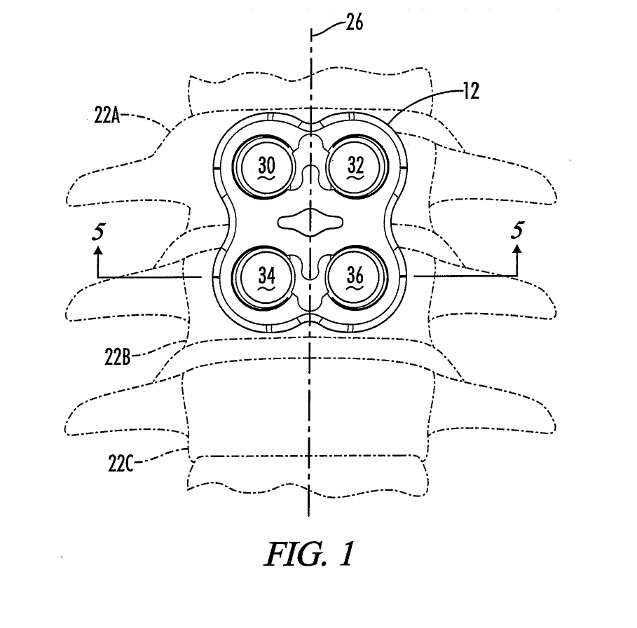

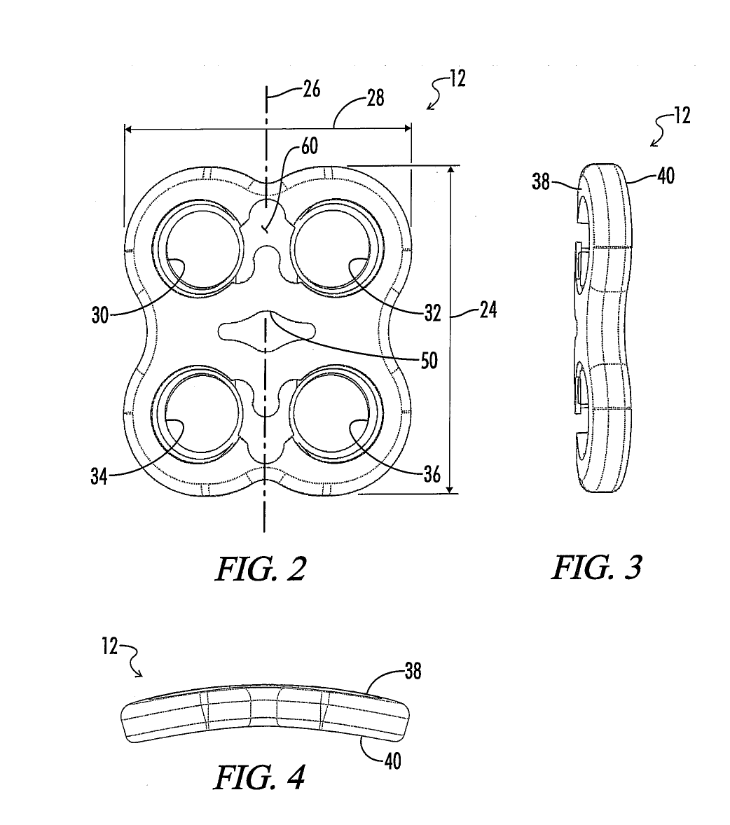

As seen in FIG. 1, the plate 12 as illustrated is constructed to overlie two adjacent cervical vertebrae 22A and 22B. As seen in FIG. 2, the cervical plate 12 has a length 24 defined generally parallel to a longitudinal axis 26. The plate 12 has a width 28 perpendicular to the longitudinal axis 26. As best seen in FIG. 1, the longitudinal axis 26 is also generally aligned with a longitudinal axis of the patient's spinal column defined along the cervical vertebrae 22A-22C.

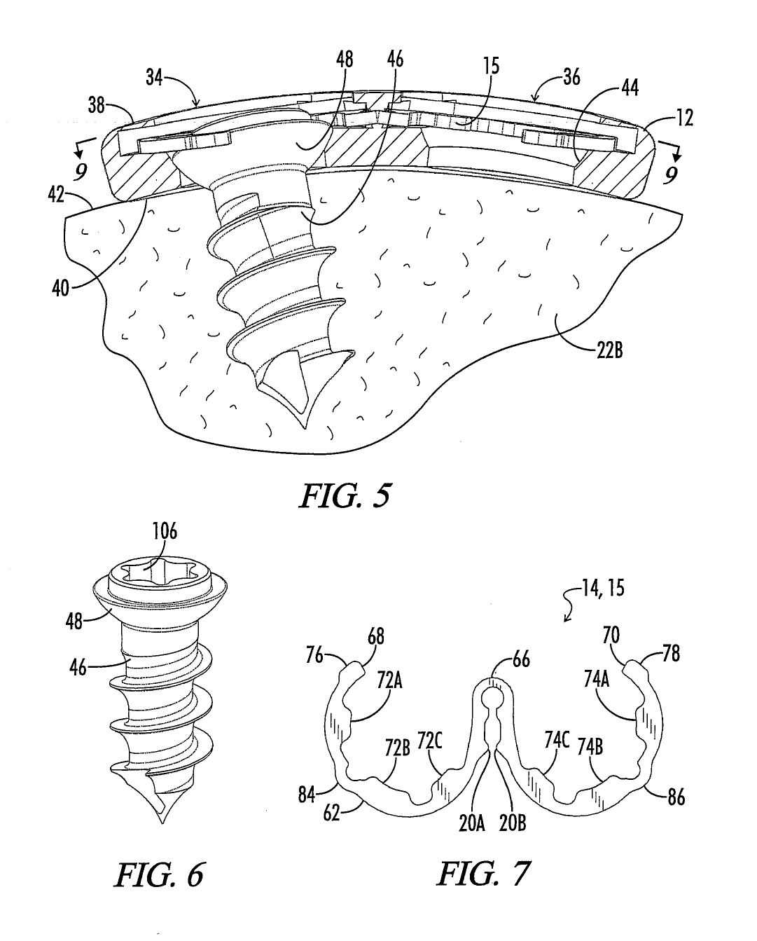

The cervical plate 12 as shown in FIG. 2 has a first pair of holes or passages 30, 32 and a second pair of holes or passages 34, 36 defined therethrough from a forward or anterior surface 38 of the plate to a rearward or posterior surface 40 of the plate. As best seen in FIGS. 4 and 5, the plate 12 has a curvature designed to conform to the curvature of a forward surface 42 of the cervical vertebrae such as 22B. The plate 12 also has a slight curvature along its length to conform generally to the curvature of the spine. The holes 30, 32, 34 and 36 may be referred to as screw receiving holes or passages through the cervical plate 12.

The hole 36 is shown in cross-sectional view in FIG. 5 where it is seen that the hole 36 has a reduced diameter socket 44 defined near its lower end. As seen in the left hand side of FIG. 5, a screw 46 is received through each of these passages and each screw 46 has an enlarged diameter head 48 on its upper end which is seated within one of the sockets when the screw is inserted through its associated hole and screwed into the bone such as 22 to attach the cervical plate 12 to the bone 22.

The holes 30 and 32 may be described as a first pair of screw receiving holes aligned perpendicularly to the longitudinal axis 26 and located on opposite sides of the longitudinal axis 26. The holes 34 and 36 may be described as a second pair of screw receiving holes axially spaced from the first set of holes 30, 32.

While the cervical plate 12 illustrated in FIG. 1 is constructed to connect the two cervical vertebrae 22A and 22B for a fusion operation in which those two vertebrae are to be fused together, it will be understood that the cervical plate 12 may be further elongated and include a third pair of holes for connection of the cervical plate to the third cervical vertebrae 22C to permit the three vertebrae to be fused together. Similarly, the cervical plate may be designed to connect four or five vertebrae.

Between the first pair of holes 30, 32 and the second pair of holes 34, 36, a graft viewing opening 50 is defined through the plate 12 for allowing the surgeon to view the placement of a cervical interbody in the disc space between the vertebrae 22A and 22B.

The cervical plate 12 includes an undercut groove at least partially surrounding each of the holes 30-36 for receipt of the associated retaining clips 14 and 15. Each of the holes 30, 32, 34 and 36 has undercut grooves 52, 54, 56 and 58, respectively, associated therewith as seen in FIG. 8. FIG. 5 shows in cross-section the hole 34 surrounded by its associated undercut groove 56. The profiles of the grooves 52, 54, 56 and 58 are shown for example in FIG. 8.

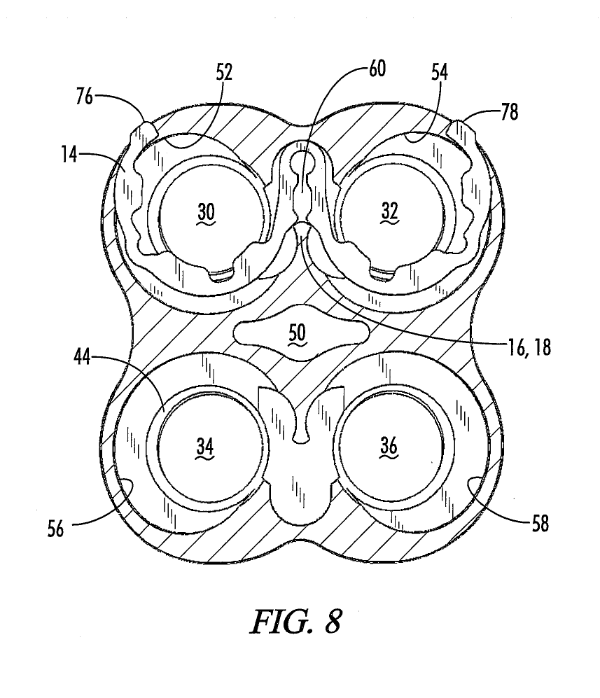

The first clip 14 is shown in a relaxed elevation view in FIG. 7. It is seen that the clip 14 may be described as generally w-shaped. The clip 14 includes a first open loop portion 62 and a second open loop portion 64 joined by an intermediate portion 66. The first open loop portion 62 has a first free end 68 and the second open loop portion 64 has a second free end 70.

The generally w-shaped clip 14 may be described as having a middle leg 66 coincident with the intermediate portion 66, and as having outer legs coincident with the outermost portions of the first and second open loop portions 62 and 64. The axial motion of the clip 14 as it moves between its actuated and unactuated positions may be described as motion in a direction parallel to the middle leg 66.

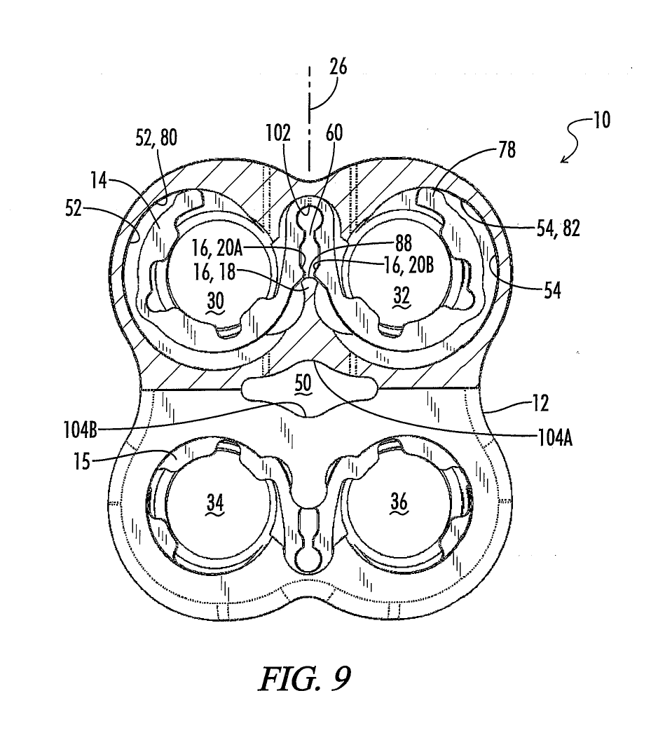

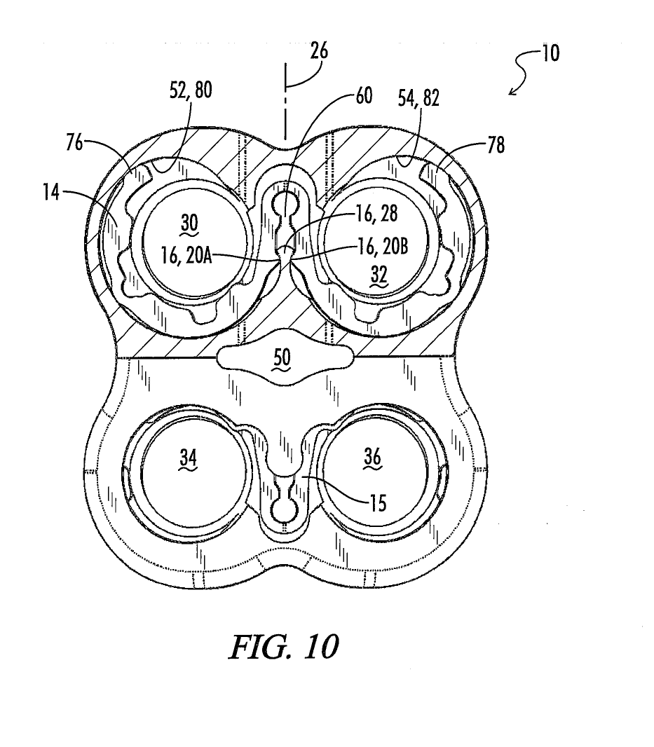

When assembled with the cervical plate 12 as shown in FIGS. 9 and 10, the clip 14 is received in the first and second undercut grooves 52 and 54 and spans the central area 60. More specifically, the first open loop portion 62 is received in the first undercut groove 52. The second open loop portion 64 is received in the second undercut groove 54. The intermediate portion 66 spans the central area 60. FIG. 9 also shows a second clip 15 in place around the lower holes 34 and 36.

The first and second open loop portions 62 and 64 may alternatively be referred to as first and second partially annular portions 62 and 64.

The first open loop portion 62 includes a plurality, preferably three, laterally inward extending interference tabs or retention tabs 72A, 72B, and 72C. Similarly, the second open loop portion 64 includes three laterally inward extending retention tabs 74A, 74B, and 74C. With reference to the retention tabs of the second open loop portion 64, the tab 74A is located generally at the 3:00 position, the tab 74B is located generally at the 5:00 position and the tab 74C is located generally at the 7:00 position. These tabs are designed to be displaced radially or laterally outward as the enlarged head 48 of the screw 46 is advanced past them, and to then spring back to their original position as the screw 46 is seated within the socket 44 of the cervical plate 12 and within the bone member 22. The associated undercut groove has sufficient clearance to allow for outward deflection of the retaining clip 14 to permit screw passage. Thus, the screw 46 is locked into place merely by the act of the surgeon installing the screw 46 with no additional locking steps required. Such a locking member may be referred to as a zero-step locking member.

As can be seen in comparing the positions of the spring clip 14 in FIGS. 8 and 9, when the spring clip 14 is confined within the profile of the undercut grooves 52 and 54 the partially open loops 62 and 64 are deflected laterally inwardly so that the outer portions thereof are resiliently biased laterally outward away from their respective holes 30 and 32.

The free ends 68 and 70 may have laterally outwardly facing cam follower tabs 76 and 78 defined thereon which are engaged with cam surface portions 80 and 82, respectively, of their associated undercut grooves 52 and 54. As can be seen in comparing FIGS. 9 and 10, when the clip 14 moves downward from its actuated position of FIG. 9 to its unactuated position of FIG. 10 relative to the cervical plate 12, the resilient bias in the open loop portions 62 and 64 causes the cam follower tabs 76 and 78 to move laterally outward as guided by their engagement with the cam surfaces 80 and 82, from the positions of FIG. 9 to the positions of FIG. 10.

As can be seen in FIG. 9, when the clip 14 is in its actuated position, the tabs 72A-72C project laterally inward to partially resiliently block the hole 30, and the tabs 74A-74C partially resiliently block the second hole 32. When the clip 14 is moved downward relative to the cervical plate 12 from the position of FIG. 9 to the position of FIG. 10, the laterally outward motion of the outer arms of the open loop portions 62 and 64 plus the downward motion of the clip 14 moves the tabs 72A-72C and 74A-74C laterally outward away from their respective holes 30 and 32 so as to allow the screws located below the clip 14 to be removed as is further described below.

With reference to FIG. 7, each of the open loop portions 62 and 64 may optionally include a strain relief bump 84 and 86, so that a major portion of the deflection of the clip will occur substantially at the strain relief bumps. The strain relief bumps 84 and 86 may be altered in position and construction or omitted depending upon the desired deflection of the clip 14.

As previously noted, the cervical plate assembly 10 includes a retention assembly 16 having first and second retention structures 18 and 20 defined on the cervical plate 12 and on the clip 14. The first locking member 18 defined on the central area 60 of the cervical plate 12 is an enlarged knob shaped protuberance 18 defined between the two undercut grooves 52 and 54. The second locking member or retention structure 20 defined on the clip 14 is comprised of a pair of bumps or detents 20A and 20B defined on the intermediate clip portion 66 and facing each other on opposite sides of the protuberance 18.

In the actuated position of FIG. 9, the bumps 20A and 20B engage an upper surface 88 of protuberance 18, and in the lower unactuated position of FIG. 10, the bumps 20A and 20B are located below the first retaining structure 18. The respective engagements of the first retention part 18 with the bumps 20A and 20B in either their upper actuated position of FIG. 9 or their lower unactuated position of FIG. 10 is maintained by the frictional engagement of the retention parts 18 and 20 when placed in either the actuated or unactuated position.

Although the detent mechanism illustrated utilizes bumps on the cervical plate interacting with bumps on the clip, alternatively the bumps on either component could work in concert with notches on the other component to form an alternative simple detent structure.

It is noted that when the clip 14 moves from its actuated position of FIG. 9 to its unactuated position of FIG. 10, it moves in an axial direction generally parallel to the longitudinal axis 26 of the clip 14.

The clip 14 is preferably integrally formed from a nitinol material. The clip 14 may for example be cut from a sheet of nitinol material. The clip 14 is thin enough to actuate within the undercut grooves 52 and 54, yet thick enough to provide adequate screw retention force when in the actuated position of FIG. 9.

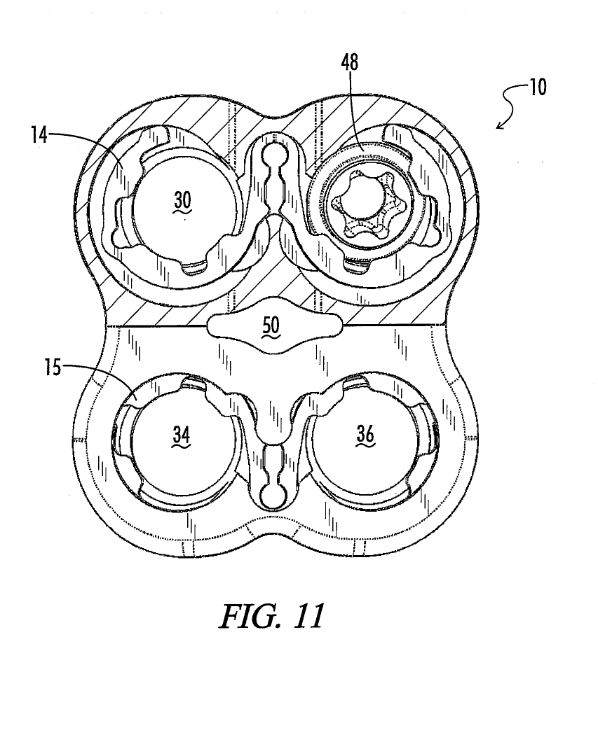

As can best be seen in FIG. 11 on the right hand side, the enlarged head 48 of the screw 46 is larger than an opening through the clip 14, defined as the space between the innermost edges of the tabs 74A-74C when the clip 14 is in the locked position as seen in FIG. 11.

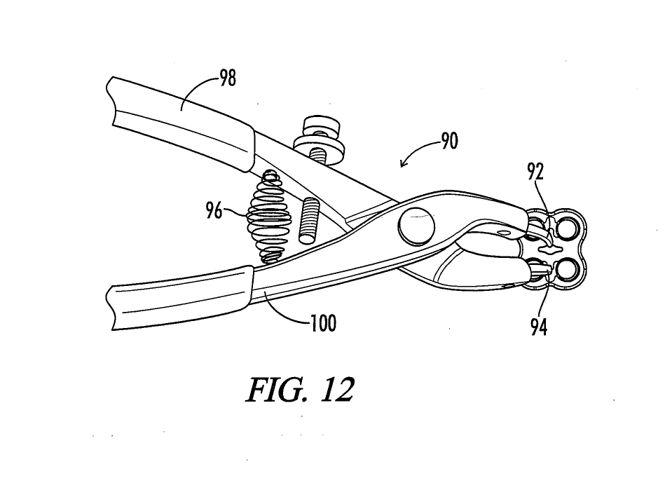

In order to move the clip 14 between its actuated position of FIG. 9 and its unactuated position of FIG. 10, a pliers-type actuating tool 90 as seen in FIG. 12 may be used. The actuating tool 90 has first and second actuating elements 92 and 94 extending therefrom which can be moved toward and away from each other by the action of the pliers. A spring element 96 is located between plier handles 98 and 100 to urge the actuating elements 92 and 94 away from each other, and the actuating elements 92 and 94 may be moved back toward each other by an operator grasping the handles 98 and 100 and squeezing the handles 98 and 100 toward each other.

As identified in FIG. 9, the actuating elements 92 and 94 of the actuating tool 90 may be received in first and second actuating tool engagement structures 102 and 104 defined on the clip 14 and the cervical plate 12, respectively. For example, the first actuating tool engagement structure 102 is seen to be a substantially circular opening defined in the intermediate portion 66 of the clip 14. The second actuating tool engagement structure 104, including features 104A and 104B, is defined as a recessed portion of the disc viewing opening 50.

The first and second actuating tool elements 92 and 94 may be placed in engagement with the first and second actuating tool engagement structures 102 and 104A and then moved toward each other to pull the clip 14 downward from its actuated position of FIG. 9 to its unactuated position of FIG. 10. Then, to move the clip 14 back to its actuated position, the first and second actuating elements 92 and 94 may be engaged with the first and second actuating tool engagement structures 102 and 104B and then spread apart to push the clip 14 back upward to its actuated position. Alternatively, both clips can be actuated simultaneously in either direction by engaging feature 102 of clip 14 and the corresponding feature of clip 15.

### Methods of Operation

The methods of use of the cervical clip apparatus 10 for connecting two cervical vertebrae such as 22A and 22B is generally as follows.

First and second clips such as 14 will be preassembled with the cervical plate 12 by the manufacturer and the clips 14 will be placed in their actuated positions as shown for example in FIGS. 9 and 11. When the clip is in the assembled actuating position, the outer arms are deflected inward by engagement of the cam surfaces with the cam follower tabs 76 and 78. The cervical plate 12 with its assembled clips is ready for use by the surgeon.

During the surgical procedure of cervical fusion, the surgeon will place the cervical plate 12 in a position substantially like that shown in FIGS. 1 and 5 wherein the cervical plate 12 is adjacent the cervical vertebrae 22A and 22B. Appropriate holes will be drilled in the bones and then the screws such as 46 will be inserted through their respective holes in the cervical plate and threaded into place within the bone as shown in FIG. 5.

As the screw 46 is threaded into the bone and is inserted past the tabs such as 74A-74C of the retaining clip 14, the clip 14 will flex laterally outwardly within the undercut groove to allow the enlarged head 48 of the screw 46 to pass to a position below the clip 14 wherein the enlarged head 48 will seat on the socket end 44 of the hole in the cervical plate 12. The clip 14 automatically snaps back into its actuated position as seen in FIGS. 9 and 11 after the enlarged screw head 48 passes past the clip, thereby preventing back-out of the screw without any additional locking action being necessary by the surgeon. Thus the clip 14 is a “zero-step” locking member.

The other screws 46 are similarly put into place within each of the holes of the cervical plate 12 to firmly attach the cervical plate 12 to the cervical vertebrae 22A and 22B so as to stabilize the position of the cervical vertebrae 22A and 22B relative to each other.

If for any reason, it is desired to remove one or more of the screws 46, that is accomplished in the following manner.

The actuating tool 90 of FIG. 12 is engaged with the clip 14 and the cervical plate 12 and the clip 14 is moved axially downward from the position of FIG. 9 to the position of FIG. 10 by a squeezing actuation of the pliers 90. The detents 20A and 20B spread apart and move downward past the detent 18 defined on the cervical plate 12 until the clip 14 reaches the position shown in FIG. 10. Then, the surgeon removes the actuating tool 90 and the clip 14 is held securely in its unactuated position of FIG. 10 by the engagement of the detent bumps 20A and 20B with the lower surface of the detent 18. Then, the surgeon may insert a second operating instrument such as a screw driver to engage the drive socket 106 defined in the upper end of the enlarged head 48 of screw 46 and the screw 46 may be removed without the surgeon having to simultaneously engage in any retracting action regarding the clip 14.

The detent mechanism 16 allows the surgeon to set the desired clip position and then remove the clip actuating instrument 90 allowing full visibility for subsequent screw removal.

### Alternative Embodiments

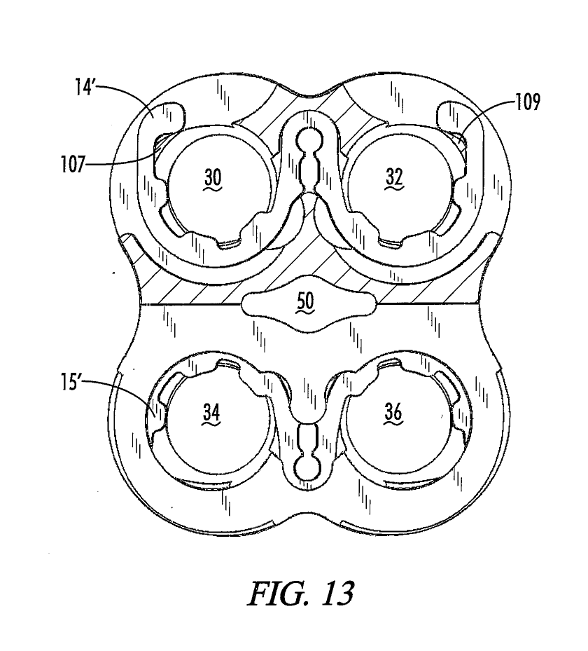

In the embodiment described above, the spring clip 14 is biased laterally outwardly when in its actuated position, and the action of cam surfaces 80 and 82 guides the outer arms of the clip 14 laterally outwardly when the clip 14 is moved from the actuated position of FIG. 9 to the unactuated position of FIG. 10. Conversely, a spring clip can be designed to be biased laterally inwardly when in its actuated position and upon selection of the unactuated position the arm could be deflected outwardly via a reversed cam arrangement. FIG. 13 schematically illustrates such an arrangement wherein laterally inwardly located cam surfaces 107 and 109 defined on the cervical plate 12 engage laterally inner surfaces of outer end portions of a modified clip 14′ so that the outer arms of clip 14′ are deflected laterally outward when the clip 14′ is moved downward relative to the actuated position of FIG. 13.

Thus it is seen that the apparatus and methods of the present invention readily achieve the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention as defined by the appended claims.

What is claimed is:

1. An orthopedic implant apparatus, comprising:
an implant body having at least one screw receiving hole defined through the implant body;
a resilient locking clip connected to the implant body and selectively movable relative to the implant body between an actuated position wherein the clip automatically prevents screw back-out from the hole after screw insertion into the hole, and an unactuated position wherein the clip is displaced relative to the hole so that the screw may be removed from the hole; and
a retention assembly including an implant retention part connected to the implant body and a clip retention part connected to the clip, the implant retention part and the clip retention part being configured to hold the clip in either selected one of the actuated and unactuated positions relative to the implant body.

2. The apparatus of claim 1, wherein:
when in the actuated position the clip partially resiliently blocks the hole.

3. The apparatus of claim 1, wherein:
the at least one hole of the implant body comprises first and second adjacent holes; and
when in the actuated position the clip partially resiliently blocks both of the first and second holes.

4. The apparatus of claim 3, wherein:
the clip includes first and second partially annular portions received about the first and second holes, respectively, and a clip intermediate portion joining the first and second partially annular portions, the clip retention part being defined on the clip intermediate portion.

5. The apparatus of claim 4, wherein:
each of the partially annular portions of the clip includes a plurality of interference tabs extending laterally inward toward the associated hole of the implant body.

6. The apparatus of claim 4, wherein:
each of the partially annular portions of the clip includes a free end resiliently biased laterally outwardly away from the associated hole of the implant body.

7. The apparatus of claim 6, wherein:
the implant body includes first and second undercut grooves defined around the first and second holes;
the first and second partially annular portions of the clip are received in the first and second undercut grooves; and
the first and second undercut grooves include first and second cam surfaces, respectively, operatively engaged with the first and second free ends, respectively, to guide the first and second partially annular portions between laterally contracted and laterally expanded positions as the clip is moved between its actuated and unactuated positions, respectively.

8. The apparatus of claim 3, wherein:
the implant body has a length defining an axial direction;
the at least one hole includes first and second holes aligned perpendicularly to the axial direction; and
the clip is displaced in the axial direction when the clip is moved between the actuated and unactuated positions.

9. The apparatus of claim 3, wherein:
the at least one hole further comprises third and fourth holes; and
further comprising a second clip operatively associated with the third and fourth holes and movable between an actuated position partially resiliently blocking the third and fourth holes, and an unactuated position.

10. The apparatus of claim 9, wherein:
the implant body further includes a graft viewing opening defined therethrough and located between the first and second clips.

11. The apparatus of claim 3, wherein the clip has a w-shape.

12. The apparatus of claim 1, further comprising:
at least one screw received through the at least one hole, the at least one screw having an enlarged screw head larger than an opening through the clip when the clip is in the actuated position.

13. The apparatus of claim 1, wherein:
the clip is formed from a nitinol material.

14. The apparatus of claim 1, wherein:
the clip includes at least three interference tabs extending laterally inward toward the hole of the implant body.

15. The apparatus of claim 1, wherein:
the implant body comprises a cervical plate for joining first and second cervical vertebrae, the plate having a length defining a longitudinal axis;
the at least one hole comprises a first pair of holes aligned perpendicular to the longitudinal axis and located on opposite sides of the longitudinal axis, and a second pair of holes axially spaced from the first pair of holes;
the first mentioned clip in its actuated position prevents screw back-out from the first pair of holes; and
the apparatus further comprises a second clip having an actuated position preventing screw back-out from the second pair of holes.

16. The apparatus of claim 1, wherein:
the clip when in its actuated position is resiliently biased in a lateral direction; and
the implant body includes a cam surface engaging the clip, and when the clip moves from its actuated to its unactuated position the cam surface guides the clip in a lateral direction.

17. The apparatus of claim 1, wherein:
the clip when in its actuated position is resiliently biased laterally outward away from the hole.

18. The apparatus of claim 1, wherein:
the clip when in its actuated position is resiliently biased laterally inward toward the hole.

19. The apparatus of claim 1, further comprising:
a first actuating tool engagement structure defined on the clip; and
a second actuating tool engagement structure defined on the implant body, so that the clip can be moved between its actuated and unactuated positions by an actuating tool engaged with the first and second actuating structures.

20. An orthopedic implant apparatus, comprising:
a plate body including:
an outward body surface;
an inward body surface;
first and second screw receiving holes extending through the plate body from the outward body surface to the inward body surface for receiving first and second screws, respectively;
a central area between the first and second holes, the central area including a first locking member defined thereon;
a first undercut groove at least partially surrounding the first hole adjacent the outward body surface; and
a second undercut groove at least partially surrounding the second hole adjacent the outward body surface; and
a resilient clip received in the first and second undercut grooves and spanning the central area, the clip including:
a first open loop portion received in the first undercut groove and including a first free end;
a second open loop portion received in the second undercut groove and including a second free end; and
an intermediate clip portion joining the first and second open loop portions and spanning the central area, the intermediate clip portion including a second locking member defined thereon and operatively engaged with the first locking member so that the clip is movable relative to the plate body between a locked position wherein the first and second open loop portions extend laterally into the first and second holes to prevent the screws from backing out of the holes and an unlocked position wherein the first and second open loop portions are displaced laterally outward from the first and second holes so that the clip does not interfere with removal of the screws.

21. The apparatus of claim 20, wherein:
each of the first and second partially open loop portions of the clip includes a plurality of interference tabs extending laterally inward toward the associated hole of the plate body.

22. The apparatus of claim 21, wherein:
each plurality of interference tabs includes at least three tabs.

23. The apparatus of claim 20, wherein:
the plate body includes first and second cam surfaces, respectively, operatively engaged with the first and second open loop portions, respectively, to guide the first and second open loop portions between laterally contracted and laterally expanded positions as the clip is moved between its locked and unlocked positions, respectively.

24. The apparatus of claim 23, wherein:
each of the open loop portions is resiliently biased laterally outward when the clip is in its actuated position; and
the first and second cam surfaces limit and guide laterally outward movement of the open loop portions away from their respective holes as the clip is moved from its actuated position to its unactuated position.

25. The apparatus of claim 23, wherein:
each of the open loop portions is resiliently biased laterally inward when the clip is in its actuated position; and
the first and second cam surfaces force the open loop portions laterally outward away from their respective holes as the clip is moved from its actuated position to its unactuated position.

26. The apparatus of claim 20, wherein:
the plate body has a length defining an axial direction;
the first and second holes are aligned perpendicularly to the axial direction; and
the clip is displaced in the axial direction relative to the plate body when the clip is moved between the locked and unlocked positions.

27. The apparatus of claim 20, wherein:
the plate body further comprises third and fourth holes; and
further comprising a second clip operatively associated with the third and fourth holes and movable between a locked position partially blocking the third and fourth holes, and an unlocked position.

28. The apparatus of claim 27, wherein:
the plate body further includes a graft viewing opening defined therethrough and located between the first and second clips.

29. The apparatus of claim 20, wherein the clip has a w-shape.

30. The apparatus of claim 20, further comprising:
first and second screws received through the first and second holes, the screws each having an enlarged screw head larger than an opening through the clip when the clip is in the locked position.

31. The apparatus of claim 20, wherein:
the clip is formed from a nitinol material.

32. The apparatus of claim 20, further comprising:
a first actuating tool engagement structure defined on the clip; and
a second actuating tool engagement structure defined on the plate body, so that the clip can be moved between its locked and unlocked positions by an actuating tool engaged with the first and second actuating structures.

33. An orthopedic implant apparatus, comprising:
an implant body having at least one screw receiving passage defined therethrough, the passage having a reduced diameter socket end;
a screw having an elongated portion and having an enlarged head configured to seat on the socket end of the passage;
a zero-step locking member connected to the implant body and operatively associated with the passage and configured to automatically lock the head of the screw in place in the passage when the head of the screw is seated on the socket end, the locking member being selectively movable to an unlocked position; and
a retention structure configured to retain the locking member in the unlocked position without further engagement of the locking member by a surgeon so that the screw can be removed after the locking member is moved to the unlocked position.

34. The apparatus of claim 33, wherein:
the implant body includes at least two of the screw receiving passages; and
the locking member is an integrally formed member and is operatively associated with both of the two screw receiving passages to automatically lock a screw in each of the passages.

35. The apparatus of claim 34, wherein;
the locking member is w-shaped having a middle leg and two outer legs, and the locking member moves relative to the implant body in a direction parallel to the middle leg when the locking member moves between its locked and unlocked positions.

36. The apparatus of claim 35, wherein:
each of the outer legs of the locking member are resiliently biased laterally outward away from the passages when the locking member is in its actuated position.

37. The apparatus of claim 36, wherein:
the implant body includes first and second cam surfaces engaged with the outer legs of the locking member and configured to move the outer legs laterally inward toward the passages when the locking member is moved from its unlocked position to its locked position.

38. The apparatus of claim 35, wherein:
each of the outer legs of the locking member are resiliently biased laterally inward toward the passages when the clip is in its actuated position.

39. The apparatus of claim 38, wherein:
the implant body includes first and second cam surfaces engaged with the outer legs of the locking member and configured to move the outer legs laterally outward away from the passages when the locking member is moved from its locked position to its unlocked position.

40. The apparatus of claim 33, further comprising:
a first actuating tool engagement structure defined on the locking member; and a second actuating tool engagement structure defined on the implant body, so that the locking member can be moved between its locked and unlocked positions by an actuating tool engaged with the first and second actuating structures.

41. A method of attaching an orthopedic implant body to a bone, the method comprising:
(a) inserting a screw through an opening of the implant body and into the bone to seat the screw on the implant body and thus attach the implant body to the bone;
(b) automatically locking the screw in the implant body during step (a) by movement of a resilient retaining clip as the screw is inserted past the retaining clip, and thereby preventing back-out of the screw, without any additional locking action being necessary by a surgeon performing the method;
(c) moving the retaining clip to an unlocked position wherein the retaining clip is held in the unlocked position by a detent structure of the implant body; and
(d) after step (c), removing the screw without the surgeon having to simultaneously retract the retaining clip.
